(19) United States
(12) Patent Application Publication
(10) Pub. No.: US 2015/0202138 A1
(43) Pub. Date: Jul. 23, 2015

(54) COSMETIC COMPOSITION

(71) Applicant: MILBON CO., LTD., Osaka (JP)

(72) Inventors: Naoto MATSUMOTO, Osaka (JP); Tsugunori HORII, Osaka (JP); Aya NISHIO, Osaka (JP)

(21) Appl. No.: 14/601,169

(22) Filed: Jan. 20, 2015

(30) Foreign Application Priority Data
Jan. 21, 2014 (JP) 2014-008539

(51) Int. Cl. A61K 8/64; A61K 8/41; A61K 8/34; A61Q 5/00

(52) U.S. Cl. CPC A61K 8/64; A61Q 5/00; A61K 8/416; A61K 8/34; A61K 2800/59

(57) ABSTRACT

Provided is a composition in which the dispersibility of a specific modified peptide is less likely to decrease at a pH of less than 7.0. The cosmetic composition contains a modified peptide having one or more side chain groups including one or more units selected from the group consisting of a structure represented by formula (Ia): —S—S—(CH₂)ₙ—COOH, wherein n is 1 or 2, a structure represented by formula (Ib): —S—S—CH(CH₃)—COOH, a structure represented by formula (Ic): —S—S—CH(COOH)—CH₂—COOH, and salts of these structures; and a long-chain alkyltrimethylammonium salt or an N-long-chain alkylpyridinium salt, wherein the ratio of the total content of the long-chain alkyltrimethylammonium salt and the N-long-chain alkylpyridinium salt to the content of the modified peptide is 0.5 or more, and the cosmetic composition has a pH of less than 7.0.

The present application claims priority benefit of Japanese Patent Application No. JP2014-008539, filed Jan. 21, 2014, and this application, including the specification, claims, drawings, and abstract, is incorporated herein by reference in its entirety for all purposes.

### TECHNICAL FIELD

The present description relates to a cosmetic composition containing a modified peptide. Although subject to multiple uses, in some embodiments the cosmetic composition is suitable for application to hair.

### INTRODUCTION

Sometimes, peptides obtained by hydrolysis of proteins are added to compositions for hair use, because they are expected to be effective in repairing and conditioning hair. Research and development has also been conducted to improve the effects of such peptides and provide an additional function for such peptides, in which peptide derivatives obtained by cationization, acylation, or silylation are known. JP 2010-132595 A and JP 2012-224572 A disclose a peptide derivative for use in hair treatment agents, in which the peptide derivative is a modified peptide obtained by introducing a carboxymethyldisulfide group or other groups into a peptide using a thioglycolate salt, a mercaptopropionate salt, a thiolactate salt, or a thiomalate salt. Such a modified peptide, which has a carboxyl group in the introduced group, has improved dispersibility in water as compared with that of the peptide before the introduction of the group.

However, the improved dispersibility of such a modified peptide is achieved at a pH of 7.0 or more, and in some cases, the dispersibility of such a modified peptide decreases with decreasing pH. In some cases, the addition of a material known as a cationic surfactant also decreases the dispersibility of such a modified peptide. In a liquid cosmetic composition containing such a modified peptide, the dispersibility of the modified peptide is more likely to significantly decrease, and even precipitation of the modified peptide occurs in some cases.

### SUMMARY

In view of these circumstances, an object of the present invention is to provide, in some embodiments, a composition in which the dispersibility of a specific modified peptide is less likely to decrease at a pH of less than 7.0.

As a result of earnest study, the inventors have found that even when a cosmetic composition containing a modified peptide has a pH of less than 7.0, the dispersibility of the modified peptide in the composition is increasable by the addition of a sufficient amount, relative to the modified protein, of a long-chain alkyltrimethylammonium salt or an N-long-chain alkylpyridinium salt.

Specifically, the present description regards a cosmetic composition, containing: a modified peptide having one or more side chain groups including one or more units selected from the group consisting of a structure represented by formula (Ia): —S—S—(CH₂)ₙ—COOH, wherein n is 1 or 2, a structure represented by formula (Ib): —S—S—CH(CH₃)—COOH, a structure represented by formula (Ic): —S—S—CH(COOH)—CH₂—COOH, and salts of these structures; and a long-chain alkyltrimethylammonium salt or an N-long-chain alkylpyridinium salt, wherein the ratio (II)/(I) is 0.5 or more, wherein (I) represents the mass of the modified peptide, and (II) represents the mass of the long-chain alkyltrimethylammonium salt and the N-long-chain alkylpyridinium salt, and the cosmetic composition has a pH of less than 7.0.

The cosmetic composition, in some embodiments, has a viscosity of less than 200 mPa·s.

In the cosmetic composition, in some embodiments, the long-chain alkyltrimethylammonium salt is a trimethylammonium salt represented by formula (IIa):

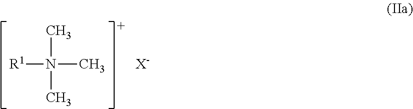

wherein R¹ represents an alkyl group having from 12 to 22 carbon atoms, and X represents a halogen atom.

In the cosmetic composition, in some embodiments, the N-long-chain alkylpyridinium salt is a pyridinium salt represented by formula (IIb):

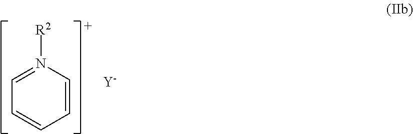

wherein R² represents an alkyl group having from 12 to 22 carbon atoms, and Y represents a halogen atom.

The cosmetic composition, in some embodiments, has a pH equal to 3.0 or more. The pH, in some embodiments, is less than 6.5. Even at a pH of less than 6.5, the dispersibility of the modified peptide is less likely to decrease.

The cosmetic composition, in some embodiments, the ratio (II)/(I) is 1.0 or more.

The cosmetic composition, in some embodiments, contains a lower dihydric alcohol or a lower trihydric alcohol. The addition of a lower dihydric or trihydric alcohol, in some embodiments, suppresses the precipitation of the modified peptide when the cosmetic composition has, e.g., a liquid form and is allowed to stand at low temperature and then returned to room temperature.

The cosmetic composition, in some embodiments, is applied to hair when used. The cosmetic composition, in some embodiments, is used to form a hair treatment agent. The hair treatment agent, in some embodiments, further comprises an amphoteric surfactant. The hair treatment agent, in some embodiments, further comprises an ester oil.

In some embodiments of the cosmetic composition, the dispersibility of the modified peptide is less likely to decrease even when the composition has a pH less than 7.0 and contains a long-chain alkyltrimethylammonium salt or an N-long-chain alkylpyridinium salt, because the content of the long-chain alkyltrimethylammonium salt and the N-long-chain alkylpyridinium salt is present in a sufficient quantity.

The cosmetic composition, in some embodiments, is in a form of a liquid cosmetic composition at normal room temperature.

### BRIEF DESCRIPTION OF THE DRAWINGS

FIG. 1 is a photograph showing cosmetic compositions of Comparative Example 1 and Examples 1a to 1d;

FIG. 2 is a photograph showing cosmetic compositions of Comparative Example 2 and Examples 2a to 2d;

FIG. 3 is a photograph showing cosmetic compositions of Examples 3a and 3b, Reference Example 1, Examples 4a and 4b, and Reference Example 2;

FIG. 4 is a photograph showing cosmetic compositions of Reference Examples 3a to 3f; and

FIG. 5 is a flow chart showing an example of a method for producing a modified peptide according to the present invention.

### DESCRIPTION OF EMBODIMENTS

A cosmetic composition according to an embodiment contains a modified peptide, a long-chain alkyltrimethylammonium salt or an N-long-chain alkylpyridinium salt, and water (for example, the content of water is 90% by mass of the cosmetic composition or more), and has a specific pH value. The cosmetic composition according to an embodiment further contains one or more optional materials used for raw materials for hair treatment agents.

### (Modified Peptide)

The cosmetic composition according to an embodiment contains a modified peptide. The modified peptide includes a main chain and a side chain group or groups bonded to the main chain, wherein the main chain includes two or more amino acids peptide-bonded together.

The main chain of the modified peptide may be of any type. The main chain is, for example, the same as that of a peptide containing cysteine as a constituent amino acid. The peptide containing cysteine as a constituent amino acid is, for example, keratin or casein. Keratin is known to have high cysteine content among naturally-derived peptides. Keratin can be used as a raw material from which the modified peptide can be efficiently obtained. From this point of view, the main chain of the modified peptide is, in some embodiments, the same as that of keratin.

The specific modified peptide has one or more side chain groups including one or more units selected from the group consisting of
a structure represented by formula (Ia): —S—S—(CH₂)ₙ—COOH,
   wherein n is 1 or 2,
a structure represented by formula (Ib): —S—S—CH(CH₃)—COOH,
a structure represented by formula (Ic): —S—S—CH(COOH)—CH₂—COOH, and
salts of these structures.

Salts of the structures represented by formulae (Ia) to (Ic) each include a carboxylate anion and a cation, which are ionically bonded together. A unit for forming the cation may be, for example, ammonium such as NH₄ or a metal atom such as Na or K.

As the molecular weight of the modified peptide decreases, its dispersibility in the composition according to an embodiment increases, and the effect of a reduction in the pH of the composition on its dispersibility decreases. On the other hand, as its molecular weight increases, its dispersibility decreases when the pH of the composition according to an embodiment is reduced. From these points of view, the modified peptide, in some embodiments, has a molecular weight of 70,000 or less, or 50,000 or less, or 30,000 or less. In some embodiments, the lower limit of the molecular weight is, but not limited to, 10,000. The molecular weight of the modified peptide may be determined as follows. The modified peptide is subjected to sodium dodecyl sulfate-polyacrylamide gel electrophoresis (SDS-PAGE). The resulting band is compared with bands of molecular weight markers. The molecular weight calculated from the resulting relative distance is assumed and used as the molecular weight of the modified peptide.

In some embodiments, the lower limit of the content of the modified peptide in the cosmetic composition according to an embodiment is, but not limited to, 0.0001% by mass of the cosmetic composition, and, in some embodiments, is 0.01% by mass of the cosmetic composition. The upper limit of the modified peptide content, in some embodiments, is 5% by mass of the cosmetic composition, or 3% by mass of the cosmetic composition, or 2% by mass of the composition, or 0.5% by mass of the cosmetic composition, in order to limit an increase in cost caused by the use of a large amount of the modified peptide and to increase the transparency of the cosmetic composition.

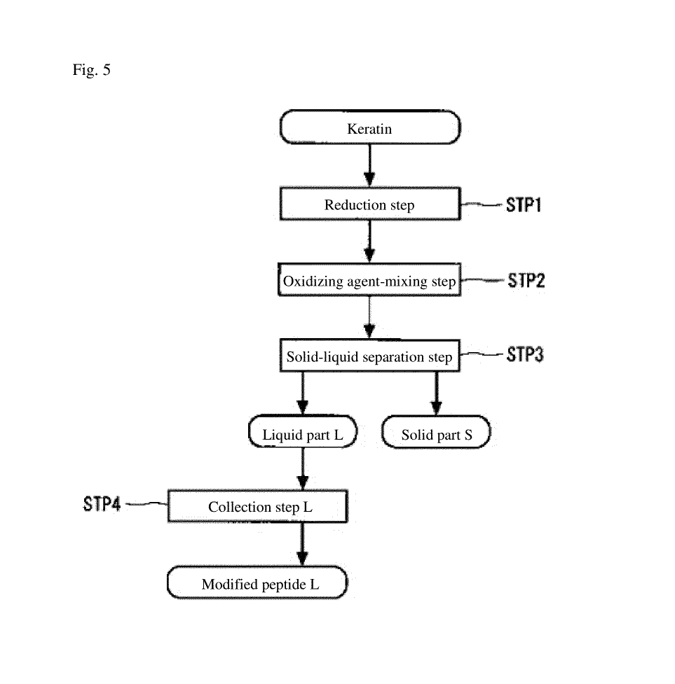

Next, a method for producing the modified peptide from keratin as a raw material will be described as an example of how to produce the modified peptide. This method is adaptable for other raw materials. As shown in FIG. 5, a method for producing the modified peptide includes a reduction step (STP1), an oxidizing agent-mixing step (STP2), a solid-liquid separation step (STP3), and a collection step L (STP4). In the method including all the steps shown in FIG. 5, the modified peptide is produced in the oxidizing agent-mixing step (STP2) (the modified peptide is dissolved in a liquid part L shown in FIG. 5 and the modified peptide is contained in a solid part S shown in FIG. 5). Therefore, the modified peptide, in some embodiments, is produced without the solid-liquid separation step (STP3) and the collection step L (STP4). Keratin:

Raw materials for keratin include wool (such as merino wool or Lincoln sheep wool), human hair, animal hair, feathers, and nails, which contain keratin as a constituent protein. In particular, to produce the modified peptide inexpensively and stably, wool is, in some embodiments, used as a raw material. Wool or other raw materials, in some embodiments, is/are subjected to a pretreatment including any appropriate combination of sterilization, degreasing, cleaning, cutting, grinding, and drying.

Reduction step (STP1):

The reduction step (STP1) includes mixing a reducing agent, keratin, and water. In the reduction step (STP1), disulfide groups (—S—S—) in keratin are reduced to mercapto groups (—SH HS—).

The reducing agent used in the reduction step (STP1), in some embodiments, is one or more agents selected from thioglycolic acid, thioglycolate salts, mercaptopropionic acid, mercaptopropionate salts, thiolactic acid, thiolactate salts, thiomalic acid, and thiomalate salts. In some embodiments, a combination of any two or more of these reducing agents is used. Examples of such a combination include, e.g., a combination of thioglycolic acid and a thioglycolate salt, a combination of two thioglycolate salts, a combination of mercaptopropionic acid and a mercaptopropionate salt, a combination of two mercaptopropionate salts, a combination of thiolactic acid and a thiolactate salt, a combination of two thiolactate salts, a combination of thiomalic acid and a thiomalate salt, a combination of two thiomalate salts, a combination of a thioglycolate salt and a mercaptopropionate salt, a combination of a thioglycolate salt and a thiolactate salt, a combination of a thioglycolate salt and a thiomalate salt, a combination of a mercaptopropionate salt and a thiolactate salt, a combination of a mercaptopropionate salt and a thiomalate salt, a combination of a thiolactate salt and a thiomalate salt, and a combination of a thioglycolate salt, a thiolactate salt, and a thiomalate salt.

Examples of thioglycolate salts include sodium thioglycolate, potassium thioglycolate, lithium thioglycolate, and ammonium thioglycolate. Examples of mercaptopropionate salts include sodium mercaptopropionate, potassium mercaptopropionate, lithium mercaptopropionate, and ammonium mercaptopropionate. Examples of thiolactate salts include sodium thiolactate, potassium thiolactate, lithium thiolactate, and ammonium thiolactate. Examples of thiomalate salts include sodium thiomalate, potassium thiomalate, lithium thiomalate, and ammonium thiomalate.

The reducing agent, in some embodiments, is used in an amount ranging from 0.005 moles to 0.02 moles based on 1 g of the raw material for keratin such as wool. The reducing agent, in some embodiments, is used in an amount ranging from 0.1 mol/L to 0.4 mol/L based on the volume of the liquid to be processed (the liquid that contains keratin or keratin-derived, processed products and is to be subjected as a reaction system to each step; hereinafter, the same applies).

In some embodiments, the amount of water is, but not limited to, 20 parts by mass to 200 parts by mass based on 1 part by mass of the raw material such as wool. In some embodiments, the water has cosmetically acceptable grade.

In the reduction step (STP1), one or more alkaline compounds, in some embodiments, are added to the liquid to be processed. The alkaline compounds are compounds that can make water alkaline when added to the water. Examples of the alkaline compounds include lithium hydroxide, sodium hydroxide, potassium hydroxide, barium hydroxide, sodium carbonate, potassium carbonate, and ammonia.

The alkaline compound, in some embodiments, is added in any amount capable of adjusting the pH of the liquid to be processed in the reduction step (STP1) to a level in the range shown below. The lower limit of the pH in the reduction step (STP1) is, in some embodiments, 9 or 10. The upper limit of the pH in the reduction step (STP1) is, in some embodiments, 13 or 12. When the pH in the reduction step (STP1) is at least the lower limit, it is possible for keratin to be efficiently reduced. When the pH is at most the upper limit, it is possible for the cleavage of the main chain of keratin to be suppressed (In some embodiments, if the cleavage of the main chain of keratin is to be promoted, the pH of the liquid to be processed is adjusted to higher than 13).

In some embodiments, the temperature of the reduction step (STP1) range from, but not limited to, 35° C. to 60° C. or 40° C. to 50° C. If the temperature is lower than 35° C., it is possible for the rate of the reducing reaction for the conversion of disulfide groups to mercapto groups to be low so that keratin may fail to be sufficiently reduced. On the other hand, if the temperature is higher than 60° C., it is possible for the main chain of keratin to be easily cleaved. The lower the setting temperature, the longer the time period of the reduction step (STP1), and the higher the setting temperature, the shorter the time period.

### Oxidizing Agent-Mixing Step (STP2):

The oxidizing agent-mixing step (STP2) includes mixing an oxidizing agent with the processed product (keratin-derived product) resulting from the reduction step (STP1) to produce a modified peptide. The oxidizing agent is added to promote an oxidation reaction for the modification of mercapto groups in the processed product. Usually, the oxidizing agent is added to the liquid to be processed, which contains the processed product resulting from the reduction step (STP1).

The oxidizing agent is, for example, sodium bromate, potassium bromate, sodium perborate, potassium perborate, hydrogen peroxide, or the like. In some embodiments, one or more oxidizing agents are used.

As a non-limiting example, in some embodiments, the oxidizing agent is used in an amount ranging from 0.001 moles to 0.02 moles based on 1 g of the raw material for keratin such as wool, or is used in an amount ranging from 0.02 mol/L to 1 mol/L based on the volume of the liquid to be processed in the oxidizing agent-mixing step (STP2).

In the process of mixing the oxidizing agent with the liquid to be processed, in some embodiments, the oxidizing agent should be inhibited from having a locally high concentration in the liquid. For this purpose, for example, a solution of about 1 mol/L to about 5 mol/L of the oxidizing agent is gradually added over 10 minutes to 6 hours regardless of whether it is added continuously or intermittently.

The amount (A) of the oxidizing agent added to the liquid with a pH of 9 or more to be processed should, in some embodiments, be larger than the amount (B) of the oxidizing agent added to the liquid with a pH of 7 to less than 9 to be processed. This can make it possible to reduce the time sufficient to produce the modified peptide. The ratio of the amount (B) of the oxidizing agent to the sum of the amounts (A) and (B) of the oxidizing agent is, in some embodiments, 20 mol % or less, or 10 mol % or less, or 5 mol % or less, or 0 mol %.

The pH of the liquid to be processed in the oxidizing agent-mixing step (STP2) is adjusted, in some embodiments, depending on the progress of this step. At the beginning of the addition of the oxidizing agent, the pH is, in some embodiments, 9 or more, or 10 or more. In some embodiments, the pH of the liquid to be processed is made to be 13 or less, or 12 or less, or 11 or less. When the pH is 9 or more, it is possible for the modified peptide to be produced with high efficiency. When the pH is 13 or less, it is possible for the cleavage of the main chain of the keratin-derived, processed product to be suppressed. In some embodiments, at the end of the oxidizing agent-mixing step (STP2), the pH is, but not limited to, about 7.

In the oxidizing agent-mixing step (STP2), in some embodiments, the time period for which the pH is kept at 9 or more is longer than the time period for which the pH is kept at 7 to less than 9. In some embodiments, the time period for which the pH is kept at 9 to 12 is longer than the time period for which the pH is kept at 7 to less than 9. In some embodiments, the time period for which the pH is kept at 10 to 11 is longer than the time period for which the pH is kept at 7 to less than 9. When such a procedure is used, it is possible for the modified peptide to be produced with higher efficiency.

The pH of the liquid to be processed is adjusted, in some embodiments, with one or more acids selected from organic and inorganic acids. Examples of organic acids include citric acid, lactic acid, succinic acid, and acetic acid. Examples of inorganic acids include hydrochloric acid and phosphoric acid. The amount of the acid to be added may be appropriately determined while the pH of the liquid to be processed is monitored. In the process of adding the acid to the liquid to be processed, if the pH of the liquid to be processed is locally lowered, it is possible for some mercapto groups in the processed product to be converted to disulfide groups. In some embodiments, the acid is gradually added to the liquid to be processed.

The temperature of the oxidizing agent-mixing step (STP2), in some embodiments, ranges from 10° C. to 60° C. or 40° C. or lower. When the temperature is controlled in this range, it is possible for the production of cystine monoxide and other by-products to be suppressed.

When thioglycolic acid or a salt thereof, mercaptopropionic acid or a salt thereof, thiolactic acid or a salt thereof, or thiomalic acid or a salt thereof is used as the reducing agent in the reduction step (STP1), the following reaction formulae for the oxidizing agent-mixing step (STP2) can be given in the order of these reducing agents.

K—CH₂—SH+HS—CH₂COO⁻→K—CH₂—S—S—CH₂COO⁻

K—CH₂—SH+HS—CH₂CH₂COO⁻→K—CH₂—S—S—CH₂CH₂COO⁻

K—CH₂—SH+HS—CH(CH₃)COO⁻→K—CH₂—S—S—CH(CH₃)COO⁻

K—CH₂—SH+HS—CH(COO⁻)CH₂COO⁻→K—CH₂—S—S—CH(COO⁻)CH₂COO⁻

K: Keratin chain

### Solid-Liquid Separation Step (STP3):

The solid-liquid separation step (STP3) includes separating the processed liquid after the oxidizing agent-mixing step (STP2) into a liquid part L and a solid part S. In some embodiments, the solid-liquid separation step (STP3) is performed using known solid-liquid separation means such as filtration, centrifugation, compression separation, sedimentation, or floatation separation. If desired, in some embodiments, desalting or the like is performed using ion exchange, electrodialysis, or other techniques.

Collection step L (STP4):

The collection step L (STP4) includes collecting a modified peptide L in the form of a solid from the liquid part L, which results from the solid-liquid separation step (STP3) and contains the modified peptide L dispersed and dissolved therein. Examples of methods for collecting the modified peptide L in the form of a solid in the collection step L (STP4) include (1) collection by freeze-drying the liquid part L, (2) collection by spray-drying the liquid part L, and (3) a process including adding an acid such as hydrochloric acid to the liquid part L to lower the pH of the liquid part L to a range from about 2.5 to about 4.0 and collecting the resulting modified peptide L precipitate. If desired, in some embodiments, the collected modified peptide L solid is subjected to washing with water or an acidic aqueous solution, drying, and other processes.

### Hydrolysis Step:

When the process by the oxidizing agent-mixing step (STP2) is completed as described above, there are obtained a modified peptide highly dispersed and dissolved in the processed liquid and a modified peptide not dissolved in the liquid. If these modified peptides are converted to low-molecular-weight peptides, it is possible for them to have improved dispersibility and solubility in water. Methods for the conversion to low-molecular-weight peptides include (1) hydrolyzing the solid part S resulting from the solid-liquid separation step (STP3), (2) hydrolyzing the modified peptide L dissolved in the liquid part L resulting from the solid-liquid separation step (STP3), (3) hydrolyzing the modified peptide L collected by the collection step L, and (4) hydrolyzing the modified peptide L and the solid part S at once. Other methods for hydrolysis to form low-molecular-weight peptides include performing hydrolysis to form low-molecular-weight peptides before the reduction step (STP1), simultaneously with the reduction step (STP1), or between the reduction step (STP1) and the oxidizing agent-mixing step (STP2).

Methods for hydrolyzing the modified peptide include (a) hydrolysis with an enzyme, (b) hydrolysis with an acid, and (c) hydrolysis with an alkali, which are known for hydrolyzing peptides.

(a) Hydrolysis with Enzyme

The enzyme for the hydrolysis is, for example, an acidic protease such as pepsin, protease A, or protease B; or a neutral or alkaline protease such as papain, bromelain, thermolysin, pronase, trypsin, or chymotrypsin.

The pH for the enzymatic hydrolysis with an acidic protease should, in some embodiments, be adjusted to 1 to 3, and the pH for the enzymatic hydrolysis with a neutral or alkaline protease should, in some embodiments, be adjusted to 5 to 11. When the pH falls within the range, it is possible for high enzyme activity to be obtained.

In the enzymatic hydrolysis, in some embodiments, the reaction temperature and the reaction time are appropriately adjusted to a range from 30° C. to 60° C. and 10 minutes to 24 hours, respectively. To stop the enzymatic hydrolysis, in some embodiments, the temperature is raised to 70° C. or higher so that the enzyme can be inactivated.

(b) Hydrolysis with Acid

The acid for use in the hydrolysis is, for example, an inorganic acid such as hydrochloric acid, sulfuric acid, phosphoric acid, nitric acid, or hydrobromic acid, or an organic acid such as formic acid or oxalic acid, which may be selected as appropriate. The hydrolysis conditions are, for example, a pH of 4 or less, a reaction temperature of 40° C. to 100° C., and a reaction time of 2 hours to 24 hours.

(c) Hydrolysis with Alkali

The alkali for use in the hydrolysis is, for example, sodium hydroxide, potassium hydroxide, lithium hydroxide, barium hydroxide, sodium carbonate, potassium carbonate, lithium carbonate, sodium silicate, or sodium borate. The hydrolysis conditions are, for example, a pH of 8.0 or more, a reaction temperature of 50° C. to 100° C., and a reaction time of 20 minutes to 24 hours.

The same or analogous method as in the collection step L (STP4), in some embodiments, is used to collect the hydrolyzed modified peptide. It should be noted that if an acid is added to lower the pH to about 2.5 to about 4.0 in the collection method, it may be difficult or impossible to collect the hydrolyzed modified peptide, because it has been converted to a low-molecular-weight peptide due to the hydrolysis. (Long-chain alkyltrimethylammonium salt and N-long-chain alkylpyridinium salt)

The cosmetic composition according to an embodiment contains a long-chain alkyltrimethylammonium salt or an N-long-chain alkylpyridinium salt or contains a long-chain alkyltrimethylammonium salt and an N-long-chain alkylpyridinium salt.

The cosmetic composition according to an embodiment may contain one or more selected from long-chain alkyltrimethylammonium salts used for raw materials for cosmetics.

A trimethylammonium salt (IIa) represented by formula (IIa) below may be added as the long-chain alkyltrimethylammonium salt.

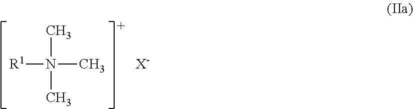

In formula (IIa), R¹ represents an alkyl group having from 12 to 22 carbon atoms (or from 12 to 20 carbon atoms, or from 12 to 18 carbon atoms), and X represents a halogen atom, such as chlorine or bromine

The long-chain alkyltrimethylammonium as a component of the trimethylammonium salt (IIa) is, for example, lauryltrimethylammonium, myristyltrimethylammonium, cetyltrimethylammonium, stearyltrimethylammonium, or behenyltrimethylammonium.

A pyridinium salt (IIb) represented by formula (IIb) below, in some embodiments, is added as the N-long-chain alkylpyridinium salt.

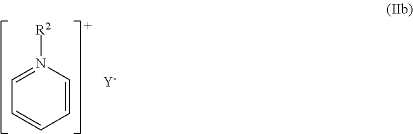

In formula (IIb), R² represents an alkyl group having from 12 to 22 carbon atoms (or from 12 to 20 carbon atoms, or from 12 to 18 carbon atoms), and Y represents a halogen atom such as chlorine or bromine

The N-long-chain alkylpyridinium as a component of the pyridinium salt (IIb) is, for example, lauryl pyridinium or cetylpyridinium.

The content of the long-chain alkyltrimethylammonium salt and the N-long-chain alkylpyridinium salt in the cosmetic composition according to an embodiment satisfies the following relationship. The ratio (II)/(I) is 0.5 or more, or 0.7 or more, or 1.0 or more, or even 1.5 or more, or 2.5 or more, wherein (I) represents the mass of the modified peptide, and (II) represents the mass of the long-chain alkyltrimethylammonium salt and the N-long-chain alkylpyridinium salt. When the ratio (II)/(I) is 0.5 or more, it is possible for the dispersibility of the modified peptide to be high, and the cosmetic composition according to an embodiment can also have high transparency. In some embodiments, the upper limit of the ratio (II)/(I) is typically, but not limited to, 300.

The content of the long-chain alkyltrimethylammonium salt and the N-long-chain alkylpyridinium salt is set at any suitable level as long as the relationship of the ratio (II)/(I) is satisfied. The content of the long-chain alkyltrimethylammonium salt and the N-long-chain alkylpyridinium salt in the cosmetic composition according to an embodiment ranges, for example, from 0.05% by mass to 3.0% by mass of the cosmetic composition.

### (Optional Materials)

The cosmetic composition according to an embodiment contains one or more optional materials, which are appropriately selected from raw materials for hair treatment agents. Such optional materials include anionic surfactants, cationic surfactants, amphoteric surfactants, fatty alcohols, polyhydric alcohols, saccharides, ester oils, oils and fats, fatty acids, hydrocarbons, waxes, silicones, high molecular compounds, amino acids, animal and plant extracts, microorganism-derived products, inorganic compounds, perfumes, preservatives, chelating agents, and ultraviolet absorbers.

The cosmetic composition according to an embodiment contains a lower dihydric alcohol or a lower trihydric alcohol or, in some embodiments, contains both a lower dihydric alcohol and a lower trihydric alcohol. When a lower dihydric or trihydric alcohol is added to the cosmetic composition according to an embodiment, it is possible for the precipitation of the modified peptide to be suppressed, which would otherwise be caused by holding the cosmetic composition at low temperature and then returning it to room temperature.

The lower dihydric alcohol has 4 or less carbon atoms, and is, for example, 1,3-butylene glycol, ethylene glycol, or propylene glycol. The lower trihydric alcohol has 4 or less carbon atoms, and is, for example, glycerin. The content of the lower dihydric alcohol and the lower trihydric alcohol in the cosmetic composition according to an embodiment ranges from 0.5% by mass of the cosmetic composition or more, or from 1% by mass or more of the cosmetic composition, or 2% by mass or more of the cosmetic composition, or even 3% by mass or more of the cosmetic composition. In some embodiments, the upper limit of the content is typically, but not limited to, 10% by mass of the cosmetic composition.

In some embodiments, the cosmetic composition is in a cosmetically acceptable medium. A cosmetically acceptable medium is a solid or liquid suitable for cosmetic formulations. In some embodiments, the cosmetically acceptable medium is or comprises water. In some embodiments, the cosmetically acceptable medium (such as water) is present in an amount greater than or equal to 90 or 95 percent by mass of the cosmetic composition. (pH)

Under conditions of 25° C., the cosmetic composition according to an embodiment has a pH of less than 7.0, which may be set at 6.5 or less, 5.0 or less, or 4.0 or less. Even when the pH is set low, the addition of the long-chain alkyltrimethylammonium salt or the N-long-chain alkylpyridinium salt makes it possible to prevent or inhibit the decrease in the dispersibility of the modified peptide and to increase the transparency of the cosmetic composition according to an embodiment. The lower limit of the pH is, for example, 3.0.

### (Viscosity)

The cosmetic composition according to an embodiment may have any level of viscosity. The viscosity is, for example, 200 mPa·s or less or 100 mPa·s or less. Even at such a low viscosity, in some embodiments, the decrease in the dispersibility of the modified peptide can be prevented or limited. The viscosity is, for example, 1 mPa·s or more. The viscosity refers to the value 60 seconds after the measurement of the viscosity is started using a Brookfield type viscometer and an appropriate rotor at 25° C. and a rotor speed of 12 rpm. Suitable viscosity measurements are taken with the viscometer model TVB-10, from TOM SANGYO CO., LTD, using Spindle (Rotor) No. M1 The undated product brochure (TOKI SANGYO CO., LTD, TVB-10 Product brochure, undated, 8 pages, the last having the number 1008/1008-51-2-2E in the lower right hand corner) of this model is or will be part of this record and is hereby incorporated by reference in its entirety.

### (Applications)

When used, the cosmetic composition according to an embodiment is applied to hair. In this case, the cosmetic composition optionally contains any or none of the above optional materials.

A cosmetic composition formulated according to an embodiment, in some embodiments, is used as a hair treatment agent. In this case, the hair treatment agent in some embodiments, is used in the form of, for example, a liquid, a cream, a wax, a gel, a solid, a foam (bubbles), or a mist.

The hair treatment agent, in some embodiments, is used as a shampoo, a rinse, a rinse-out conditioner, a leave-in conditioner, a component for a multi-agent conditioner, a preparation for permanent waving pretreatment, a preparation for permanent waving post-treatment, a preparation for coloring pretreatment, a preparation for coloring post-treatment, a preparation for bleaching pretreatment, a preparation for bleaching post-treatment, a preparation for straightening pretreatment, a preparation for straightening post-treatment, or a hairdressing agent.

Raw materials for use in the production of the hair treatment agent are, in some embodiments, selected from raw materials for hair treatment agents. Such raw materials include anionic surfactants, cationic surfactants, amphoteric surfactants, fatty alcohols, polyhydric alcohols, saccharides, ester oils, oils and fats, fatty acids, hydrocarbons, waxes, silicones, high molecular compounds, amino acids, animal and plant extracts, microorganism-derived products, inorganic compounds, perfumes, preservatives, chelating agents, and ultraviolet absorbers.

### EXAMPLES

Hereinafter, embodiments of the present cosmetic composition will be more specifically described with reference to examples, which, however, should not be construed as limiting the scope of present invention as claimed herein. (Solution of modified peptide (Ia))

The reduction step, the oxidizing agent-mixing step, the solid-liquid separation step, the collection step, the hydrolysis step, and the preparation step described below were performed so that a transparent solution was obtained in which a modified peptide (Ia) having a side chain group represented by formula (Ia) (n=1) was dispersed.

### Reduction Step

Merino wool, which had been cleaned with a neutral detergent and dried, was cut into about 5 mm pieces. A mixture of 5.0 parts by mass of the wool pieces, 15.4 parts by mass of a 30% by mass sodium thioglycolate aqueous solution, and 8.5 parts by mass of a 6 mol/L sodium hydroxide aqueous solution was prepared, and water was further mixed, so that a liquid to be processed was obtained, which had a total amount of 150 parts by mass and a pH of 11. The liquid to be processed was stirred under the conditions of 45° C. for 1 hour. Water was then further added to make up a total amount of 200 parts by mass. The resulting liquid was allowed to stand under the conditions of 45° C. for 2 hours and then allowed to naturally cool until the liquid temperature reached room temperature.

### Oxidizing Agent-Mixing Step:

After the reduction step, 178 parts by mass of an aqueous solution containing 15.26 parts by mass of 35% by mass hydrogen peroxide water was added with stiffing over about 30 minutes to the liquid to be processed (as the hydrogen peroxide water was added, the pH of the liquid increased, in which an about 20% by mass acetic acid aqueous solution was added so that the pH was adjusted to fall within the range of 10 to 11). Subsequently, an about 20% by mass acetic acid aqueous solution was gradually added so that the pH of the liquid was gradually lowered from 11 to 7.

### Solid-Liquid Separation Step and Collection Step:

The liquid resulting from the oxidizing agent-mixing step was filtered so that the insoluble product was removed from the liquid. Subsequently, 160 parts by mass of an aqueous solution containing 97.2 parts by mass of a 36% by mass hydrochloric acid aqueous solution was added to the collected liquid part (filtrate). As a result, the pH of the liquid was lowered from 7 to 3.8, so that a modified peptide (Ia) was allowed to precipitate. The precipitate was collected and washed with water to give the modified peptide (Ia) as a solid.

### Hydrolysis Step:

An aqueous solution of the solid of the modified peptide (Ia) resulting from the collection step was prepared, to which 2-amino-2-methyl-1-propanol was added to adjust its pH to 10.5. The resulting aqueous solution was heated at 80° C. for 2 hours. After the heating, the liquid was filtered to give a filtrate.

### Preparation Step:

The filtrate resulting from the hydrolysis step was mixed with phenoxyethanol, 1,3-butylene glycol, and water to form a modified peptide (Ia) solution. The solution contained 1% by mass of the modified peptide (Ia), 1% by mass of phenoxyethanol, and 3% by mass of 1,3-butylene glycol. SDS-PAGE showed that the band of the modified peptide (Ia) corresponded to a molecular weight of 44,000.

### Comparative Example 1 and Examples 1a to 1d

As shown in Table 1 below, cosmetic compositions of Comparative Example 1 and Examples 1a to 1d were prepared by mixing the modified peptide (Ia) solution, cetyltrimethylammonium chloride, and water and adjusting the pH with citric acid.

TABLE 1

| | | Compar- | | | | |
|---|---|---|---|---|---|---|
| | | ative | Exam- | Exam- | Exam- | Exam- |
| | | Exam- | ple | ple | ple | ple |
| | | ple 1 | 1a | 1b | 1c | 1d |
| (I) | Modified peptide | 0.1 | 0.1 | 0.1 | 0.1 | 0.1 |
| | (% by mass) | | | | | |
| (II) | Cetyltrimeth- | 0.03 | 0.06 | 0.09 | 0.12 | 0.15 |
| | ylammonium | | | | | |
| | chloride (% by | | | | | |
| | mass) | | | | | |
| | Stearyltrimethyl | — | — | — | — | — |
| | ammonium | | | | | |
| | chloride (% by | | | | | |
| | mass) | | | | | |

| | | | | | |
|---|---|---|---|---|---|
| Mixing ratio (II)/(I) | 0.3 | 0.6 | 0.9 | 1.2 | 1.5 |
| pH | 6.0 | 6.0 | 5.9 | 5.7 | 5.7 |

The cosmetic compositions of Comparative Example 1 and Examples 1a to 1d were visually observed (FIG. 1 is a photograph showing the cosmetic compositions of Comparative Example 1 and Examples 1a to 1d). The compositions of Examples 1a to 1d, in which the mixing ratio (II)/(I) was more than 0.5, had higher dispersibility of the modified peptide (Ia) and higher transparency than the composition of Comparative Example 1. It has been demonstrated that at a pH of 6.0, the dispersibility of the modified peptide (Ia) was lower in a composition containing 0.03% by mass of cetyltrimethylammonium chloride than in a composition containing no cetyltrimethylammonium chloride.

### Comparative Example 2 and Examples 2a to 2d

As shown in Table 2 below, cosmetic compositions of Comparative Example 2 and Examples 2a to 2d were prepared by mixing the modified peptide (Ia) solution, stearyltrimethyl ammonium chloride, and water and adjusting the pH with citric acid.

TABLE 2

| | | Compar- | | | | |
|---|---|---|---|---|---|---|
| | | ative | Exam- | Exam- | Exam- | Exam- |
| | | Exam- | ple | ple | ple | ple |
| | | ple 2 | 2a | 2b | 2c | 2d |
| (I) | Modified peptide | 0.1 | 0.1 | 0.1 | 0.1 | 0.1 |
| | (% by mass) | | | | | |
| (II) | Cetyltrimeth- | — | — | — | — | — |
| | ylammonium | | | | | |
| | chloride (% by | | | | | |
| | mass) | | | | | |
| | Stearyltrimethyl | 0.03 | 0.07 | 0.10 | 0.13 | 0.16 |
| | ammonium | | | | | |
| | chloride (% by | | | | | |
| | mass) | | | | | |

| | | | | | |
|---|---|---|---|---|---|
| Mixing ratio (II)/(I) | 0.3 | 0.7 | 1.0 | 1.3 | 1.6 |
| pH | 6.3 | 6.2 | 6.2 | 5.7 | 5.6 |

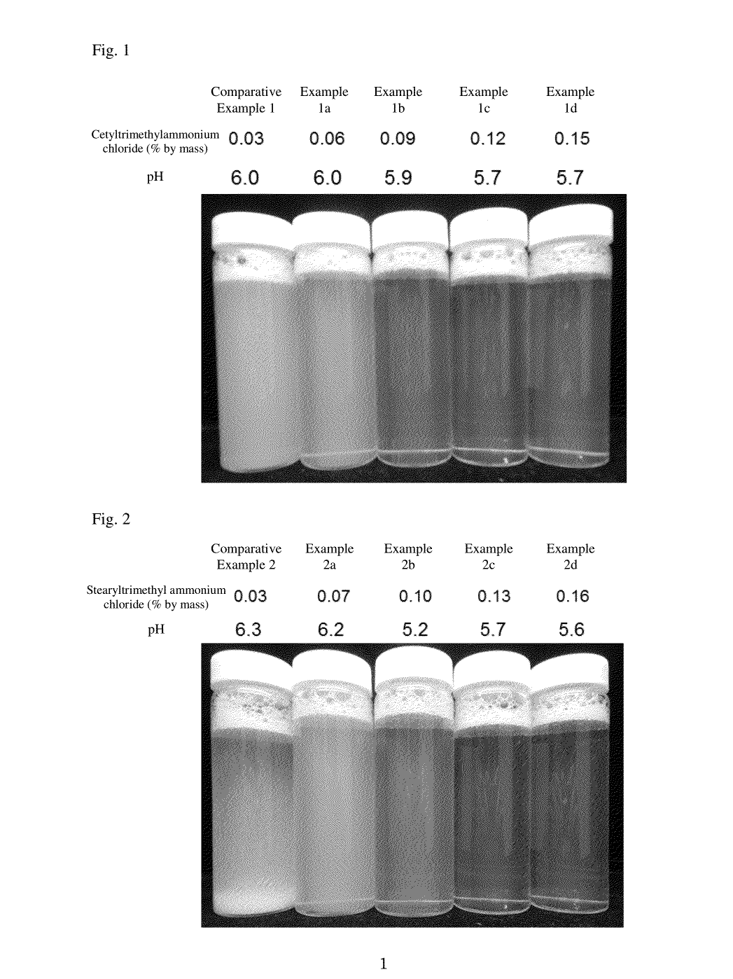

The cosmetic compositions of Comparative Example 2 and Examples 2a to 2d were visually observed (FIG. 2 is a photograph showing the cosmetic compositions of Comparative Example 2 and Examples 2a to 2d). The compositions of Examples 2a to 2d, in which the mixing ratio (II)/(I) was more than 0.5, had higher dispersibility of the modified peptide (Ia) and higher transparency than the composition of Comparative Example 2. It has been demonstrated that at a pH of 6.3, the dispersibility of the modified peptide (Ia) was lower in a composition containing 0.03% by mass of stearyltrimethyl ammonium chloride than in a composition containing no stearyltrimethyl ammonium chloride.

### Examples 3a and 3b, Reference Example 1, Examples 4a and 4b, and Reference Example 2

As shown in Tables 3 and 4 below, cosmetic compositions were prepared by mixing the modified peptide (Ia) solution, cetyltrimethylammonium chloride or stearyltrimethyl ammonium chloride, and water and adjusting the pH with citric acid or arginine.

TABLE 3

| | | | | Reference |
|---|---|---|---|---|
| | | Example 3a | Example 3b | Example 1 |
| (I) | Modified peptide | 0.1 | 0.1 | 0.1 |
| | (% by mass) | | | |
| (II) | Cetyltrimethylammonium | 0.09 | 0.09 | 0.09 |
| | chloride (% by mass) | | | |
| | Stearyltrimethyl ammonium | — | — | — |
| | chloride (% by mass) | | | |

| | | | |
|---|---|---|---|
| Mixing ratio (II)/(I) | 0.9 | 0.9 | 0.9 |
| pH | 3.5 | 5.9 | 9.0 |

TABLE 4

| | | | | Reference |
|---|---|---|---|---|
| | | Example 4a | Example 4b | Example 2 |
| (I) | Modified peptide | 0.1 | 0.1 | 0.1 |
| | (% by mass) | | | |
| (II) | Cetyltrimethylammonium | — | — | — |
| | chloride (% by mass) | | | |
| | Stearyltrimethyl ammonium | 0.13 | 0.13 | 0.13 |
| | chloride (% by mass) | | | |

| | | | |
|---|---|---|---|
| Mixing ratio (II)/(I) | 1.3 | 1.3 | 1.3 |
| pH | 5.7 | 5.6 | 9.0 |

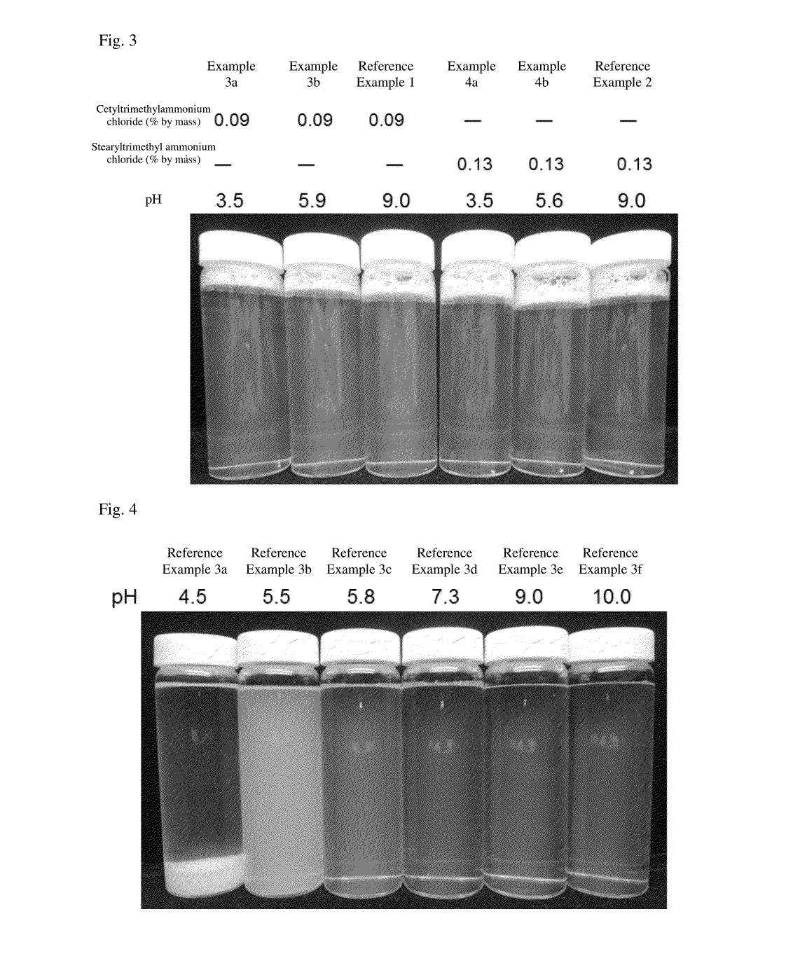

The cosmetic compositions of Examples 3a and 3b, Reference Example 1, Examples 4a and 4b, and Reference Example 2 were visually observed (FIG. 3 is a photograph showing the cosmetic compositions of Examples 3a and 3b, Reference Example 1, Examples 4a and 4b, and Reference Example 2). There was no difference in transparency between the composition of Reference Example 1 with a pH of 9.0 and the compositions of Examples 3a and 3b with a pH of less than 7.0. The dispersibility of the modified peptide (Ia) in the compositions of Examples 3a and 3b was substantially the same as in the composition of Reference Example 1. The same result was obtained for the compositions of Examples 4a and 4b and Reference Example 2.

### Reference Examples 3a to 3f

The modified peptide (Ia) solution and water were mixed, and the pH of the mixture was adjusted with citric acid or arginine. In Reference Examples 3a, 3b, 3c, 3d, 3e, and 3f, the pH was adjusted to 4.5, 5.5, 5.8, 7.3, 9.0, and 10.0, respectively. In Reference Examples 3a to 3f, neither a long-chain alkyltrimethylammonium salt nor an N-long-chain alkylpyridinium salt was added.

The cosmetic compositions of Reference Examples 3a to 3f were visually observed (FIG. 4 is a photograph showing the cosmetic compositions of Reference Examples 3a to 3f). At a pH of less than 7.0, a gradual decrease in the dispersibility of the modified peptide (Ia) was observed.

### Example 6

Cetylpyridinium chloride was gradually added to 100 parts by mass of a modified peptide solution with the same composition as above (1% by mass of the modified peptide, 1% by mass of phenoxyethanol, 3% by mass of 1,3-butylene glycol, and 95% by mass of water). It was observed that the transparency of the modified peptide solution (the dispersibility of the modified peptide) decreased from the start of the addition, but the transparency increased as the addition continued. The measured pH was 5.9 when the added amount reached 0.12 parts by mass. Subsequently, citric acid was added until the pH reached 3.5, during which the transparency remained unchanged.

Hair treatment agents of Examples 6a to 6f for use as leave-in treatments were prepared by mixing raw materials belonging to group A in Table 5 with raw materials belonging to group B in Table 5. After being frozen at −10° C. and then returned to room temperature, each hair treatment agent was visually observed, and it was determined whether or not the modified peptide (Ia) precipitated. The results are shown in Table 5 below. It was found that the precipitation of the modified peptide (Ia) was suppressed by adding a lower dihydric or trihydric alcohol or increasing the added amount of a lower dihydric or trihydric alcohol. This indicates that the addition of the lower alcohol is advantageous in suppressing the precipitation of modified peptides.

TABLE 5

| | | Example 6a | Example 6b | Example 6c | Example 6d | Example 6e | Example 6f |
|---|---|---|---|---|---|---|---|
| A | 1,3-Butylene glycol | 3 | 5 | 0.2 | 0.2 | 0.2 | 0.2 |
| | Glycerin | — | — | 3 | 5 | — | — |
| | Polyoxyethylene | 0.1 | 0.1 | 0.1 | 0.1 | 0.1 | 0.1 |
| | hardened castor oil | | | | | | |
| | (60E.O.) | | | | | | |
| | Lauryl | 0.2 | 0.2 | 0.2 | 0.2 | 0.2 | 0.2 |
| | dimethylaminoacetic acid | | | | | | |
| | betaine | | | | | | |
| | Ethanol | 5 | 5 | 5 | 5 | 8 | 10 |
| | Perfume | 0.2 | 0.2 | 0.2 | 0.2 | 0.2 | 0.2 |
| B | Citric acid | 0.1 | 0.1 | 0.1 | 0.1 | 0.1 | 0.1 |
| | Cetylpyridinium chloride | 0.3 | 0.3 | 0.3 | 0.3 | 0.3 | 0.3 |
| | Modified peptide | 3 (0.03) | 3 (0.03) | 3 (0.03) | 3 (0.03) | 3 (0.03) | 3 (0.03) |
| | solution (modified | | | | | | |
| | peptide) | | | | | | |
| | Methylisothiazoline | 0.005 | 0.005 | 0.005 | 0.005 | 0.005 | 0.005 |
| | Diethoxyethyl succinate | 5 | 5 | 5 | 5 | 5 | 5 |
| | Water | 83 | 81 | 83 | 81 | 83 | 81 |

| | | | | | | |
|---|---|---|---|---|---|---|
| After freezing and thawing | Transparent | Transparent | Transparent | Transparent | Precipitation | Precipitation |

Units for content: parts by mass

What is claimed is:

1. A cosmetic composition, comprising:
a modified peptide having one or more side chain groups comprising one or more units selected from the group consisting of
a structure represented by formula (Ia): —S—S—(CH₂)ₙ—COOH,
wherein n is 1 or 2,
a structure represented by formula (Ib): —S—S—CH(CH₃)—COOH,
a structure represented by formula (Ic): —S—S—CH(COOH)—CH₂—COOH, and
salts of these structures; and
a long-chain alkyltrimethylammonium salt or an N-long-chain alkylpyridinium salt, wherein
the ratio (II)/(I) is 0.5 or more, wherein (I) represents the mass of the modified peptide, and (II) represents the total mass of the long-chain alkyltrimethylammonium salt and the N-long-chain alkylpyridinium salt, and
the cosmetic composition has a pH of less than 7.0.

2. The cosmetic composition according to claim 1, which is liquid of a viscosity of less than 200 mPa·s.

3. The cosmetic composition according to claim 1, wherein the long-chain alkyltrimethylammonium salt is a trimethylammonium salt represented by formula (IIa): 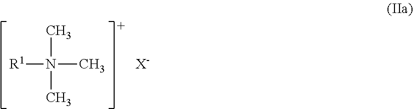
wherein R¹ represents an alkyl group having from 12 to 22 carbon atoms, and X represents a halogen atom.

4. The cosmetic composition according to claim 2, wherein the long-chain alkyltrimethylammonium salt is a trimethylammonium salt represented by formula (IIa): 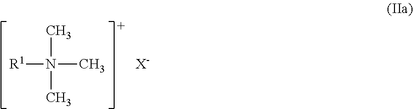
wherein R¹ represents an alkyl group having from 12 to 22 carbon atoms, and X represents a halogen atom.

5. The cosmetic composition according to claim 1, wherein the N-long-chain alkylpyridinium salt is a pyridinium salt represented by formula (IIb): 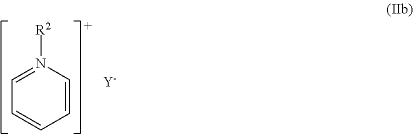
wherein R² represents an alkyl group having from 12 to 22 carbon atoms, and Y represents a halogen atom.

6. The cosmetic composition according to claim 2, wherein the N-long-chain alkylpyridinium salt is a pyridinium salt represented by formula (IIb): 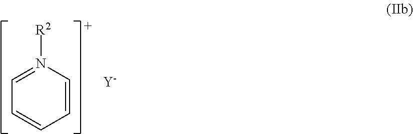
wherein R² represents an alkyl group having from 12 to 22 carbon atoms, and Y represents a halogen atom.

7. The cosmetic composition according to claim 3, wherein the N-long-chain alkylpyridinium salt is a pyridinium salt represented by formula (IIb): 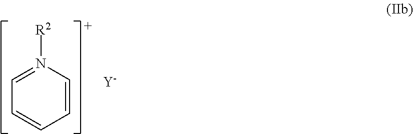
wherein R² represents an alkyl group having from 12 to 22 carbon atoms, and Y represents a halogen atom.

8. The cosmetic composition according to claim 1, wherein the pH is from 3.0 to less than 6.5.

9. The cosmetic composition according to claim 2, wherein the pH is from 3.0 to less than 6.5.

10. The cosmetic composition according to claim 3, wherein the pH is from 3.0 to less than 6.5.

11. The composition according to claim 1, wherein the ratio (II)/(I) is 1.0 or more.

12. The cosmetic composition according to claim 1, further comprising a lower dihydric alcohol or a lower trihydric alcohol.

13. The cosmetic composition according to claim 2, further comprising a lower dihydric alcohol or a lower trihydric alcohol.

14. The cosmetic composition according to claim 3, further comprising a lower dihydric alcohol or a lower trihydric alcohol.

15. The cosmetic composition according to claim 1, which is to be applied to hair.

16. A hair treatment agent comprising the cosmetic composition according to claim 1.

17. A hair treatment agent comprising the cosmetic composition according to claim 2.

18. A hair treatment agent comprising the cosmetic composition according to claim 3.

19. The hair treatment agent according to claim 18, further comprising amphoteric surfactant.

20. The hair treatment agent according to claim 18, further comprising ester oil.
